(19) United States
(12) Patent Application Publication
(10) Pub. No.: US 2019/0254546 A1
(43) Pub. Date: Aug. 22, 2019

(54) NEURAL MULTIELECTRODE ARRAYS AND THEIR MANUFACTURE AND USE

(71) Applicant: General Electric Company, Schenectady, NY (US)

(72) Inventors: Craig Patrick Galligan, Schenectady, NY (US); Nancy Cecelia Stoffel, Schenectady, NY (US); Eric Patrick Davis, Wynantskill, NY (US); Kaustubh Ravindra Nagarkar, Clifton Park, NY (US)

(21) Appl. No.: 15/902,841

(22) Filed: Feb. 22, 2018

(51) Int. Cl. A61B 5/04; A61B 5/00; A61N 1/05

(52) U.S. Cl. CPC A61B 5/04001; A61B 5/685; A61B 2562/046; A61B 2562/125; A61N 1/0551

(57) ABSTRACT

The present approach relates to the fabrication of probes of a probe array device using wire bonding techniques. In certain implementation, a wire bond apparatus bonds ones end of a wire to a region of a probe array substrate. The second end, however, is not bonded to the substrate and instead is either fabricated to be vertical with respect to the substrate or raised from a non-bonded site to be vertical. The process may be repeated to form multiple probes of the probe array.

### TECHNICAL FIELD

Aspects of the present approach generally relate to the use and/or fabrication of multielectrode probe arrays, and more particularly, to multielectrode probe arrays suitable for monitoring neural activity and/or stimulating neurons.

### BACKGROUND

Understanding the mechanisms implicated in, or otherwise related to, neural activity may be relevant to various clinical and/or research endeavors. By way of example, study of the mechanisms related to mediating learning and other forms of cortical plasticity at the level of neuronal ensembles could aid in the development of therapies for neurodegenerative disease as well as the design of assistive brain-computer interfaces.

Study of neural activity typically relies on invasive probes or probe arrays. Such probe arrays may also be of interest in the context of neural modulation or stimulation, which may be useful in various treatments or diagnostic approaches. The neural probe arrays typically have narrow, relatively long length structures (i.e., probes) capable of being effectively inserted into tissue with minimal damage and/or displacement of tissue. Manufacture of suitably narrow probes having sufficient length and durability can be problematic.

### BRIEF DESCRIPTION

In one embodiment, a method of fabricating a probe of a probe array device is provided. In accordance with this method, a wire bonding tool through which a wire passes is lowered toward a bonding pad of a probe array substrate. A ball bond is formed between an end of the wire and the bonding pad. The wire bonding tool is moved to a second region of the probe array substrate so that the wire extends from the ball bond to the second region.

The wire is deformed at the second region to form a thinned region of the wire. The wire bonding tool is moved so that the wire is at a substantially vertical orientation with respect to the bonding pad. The wire bonding tool is lifted so as to break the wire at the thinned region to form the probe of the probe array device.

In a further embodiment, a method of fabricating a probe of a probe array device is provided. In accordance with this method, a wire bonding tool through which a wire passes is lowered toward a bonding pad of a probe array substrate. A ball bond is formed between an end of the wire and the bonding pad. The wire bonding tool is moved away from the substrate in a direction perpendicular to a surface of the substrate on which the binding pad is located so that the wire extends outward perpendicular from the ball bond. A break is formed in the wire to form a probe of a probe array, wherein the location of the break corresponds to a probe tip of the probe.

In an additional embodiment, a neural probe array is provided. In accordance with this embodiment, the neural probe array comprises: a probe array substrate; a plurality of bonding pads formed on the probe array substrate; and, on each bonding pad, a respective wire bonded to each respective bonding pad to form a probe. Each probe is oriented substantially perpendicular to the probe array substrate.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts a block diagram of a neural probe array in conjunction with a monitoring and/or stimulation system, in accordance with aspects of the present approach;

FIG. 2 depicts a process flow visually depicting steps of fabricating a probe using a wire bonding approach, in accordance with aspects of the present approach;

FIG. 3 depicts an alternative process flow visually depicting steps of fabricating a probe using a wire bonding approach, in accordance with aspects of the present approach;

FIG. 4 depicts a scanning electron micrograph of a probe array fabricated in accordance with aspects of the present approach;

FIG. 5 depicts a scanning electron micrograph of a probe and probe tip fabricated in accordance with aspects of the present approach; and

FIG. 6 depicts a block diagram of a kit for use in a medical procedure including a probe array in accordance with aspects of the present approach.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements.

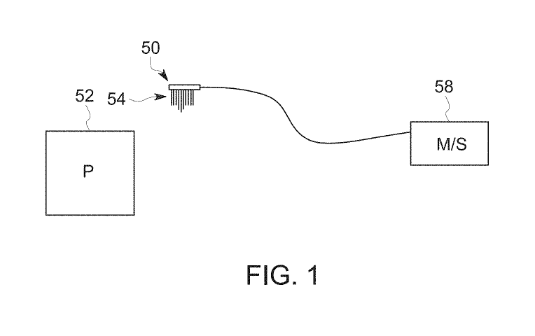

Turning to FIG. 1, neural probe arrays 50 may be of interest both for monitoring neural activity as well as for stimulating or modulating neural tissue (e.g., neural tissue of a patient 52), such as for treatment or diagnosis of various neurological disorders. Such neural probes arrays 50 typically comprise an array (e.g., a 10×10 array) of probes 54 or probe needles that are narrow enough to be inserted with minimal damage and/or displacement of tissue, while being long enough to reach the target depths for monitoring and/or treatment, while also being sturdy enough to withstand the insertion process. In accordance with one embodiment, the wires of the probe array 50 are oriented and attached generally perpendicular to the surface of the probe array substrate (i.e., “vertically” relative to the array surface. The probe array substrate may be a semiconductor wafer (such as a silicon substrate), a ceramic substrate, or a glass or polymeric substrate (including polyimide, polytetrafluoroethylene, thermoplastic polyurethane), or any other suitable substrate material. In one embodiment, the attachment surface of the substrate is metalized to facilitate attachment of the probe structures, as discussed herein.

In the present context, each probe 54 is conductive along at least a portion of its length, typically at the tip of the probe 54. Via this conductive portion the probe 54 may be used to monitor electrical activity in the inserted tissue, to provide an electrical stimulus to the inserted tissue, or a combination of these actions. To that end, FIG. 1 depicts that the probes 54, via the probe array structure 50, are connected to a monitor and/or stimulator device 58 capable of reading or providing electrical signals to each probe 54 individually or collectively.

With the preceding in mind, the present approach relates to fabricating a multi-electrode array, such as a neural probe array 50, capable of addressing these issues and performing these functions. In general, a probe array 50 manufactured in accordance with the present approach comprises multiple high aspect ratio (e.g., the ratio of length to thickness or width) probes that each have one or more exposed conductive electrode surfaces. The electrodes can be used to detect electrical signals (neural spikes) in surrounding local regions of tissue (e.g., surrounding local regions of the brain) and/or to electrically stimulate neural tissue or other tissue susceptible to such electrical stimulation.

A probe array 50 in accordance with the present approach is constructed from fine gauge metal wire, such as gold or platinum wire. In accordance with one implementation of the present approach, a probe 54 may be from 10 μm to 75 μm (such as from 18 μm to 25 μm) in diameter taking into account manufacturing tolerances and variation and from 50 μm to 1 cm (such as from 1 mm to 3 mm) long (i.e., in height). As depicted in FIG. 1, certain of the probes 54 may differ in length, which may facilitate an insertion process of the probe into tissue by reducing or eliminating the tenting effect that might otherwise be observed. In addition, the probe 54 is sufficiently mechanically stable to facilitate insertion into tissue. In certain embodiments the tip of the probe 54 is wedge-shaped or conically-shaped and reduces the insertion force needed to implant the probe array 50 into tissue. A conical tip may be formed as a consequence of the wire-bonding fabrication process discussed herein or through etching of the terminal end of the probe 54.

In a further aspect, the probe tips may have sensing functionality and may be made to be receptive to chemicals or markers of interest, such as neurotransmitters, by coating the probe tips with a functionalized polymer, oligomer, or short chain material such as, but not limited to, a functionalized silane, thiols or titanate coupling agent. Other possible coating include ionic membranes, ionophores (for detection of ionic compounds), and/or enzymes (for detection of targeted biomolecules). Additionally polycrystalline diamond or other carbon coatings may also be used for chemical detection. Coatings may also be employed to modify the electrical properties of the probe tip. For example. The impedance of the probe tip can be reduced by use of coatings including conductive polymers such as poly(3,4-ethylene dioxthiophene (PEDOT) and poly (styrene sulfonate) (PSS)), carbon-nanotubes or other nano-textured surfaces, or other compound metal alloy surfaces such as titanium nitride (TiN), platinum iridium, and iridium oxide.

As discussed herein, the probes 54 have isolated areas of exposed electrically conductive material, such as at the tip of each probe 54. These conductive regions are electrically connected to the base substrate and may interface with external components for analysis, multiplexing, and/or recording. For example, in one implementation, the probes 54 have exposed gold at the tip and that is the only region that is electrically functionalized. By way of example, in one implementation, the wire used to form the probe 54 may be a dielectric-coated gold wire bonding wire, which offers electrical isolation where the coating is present, such as along the length of the shaft of the probe 54. Thus, in such an implementation the dielectric serves to limit the electrically conductive stimulation/sensing region to only the tip of the probe 54. Other approaches for achieving this electrical isolation along the shaft of the probe wire that may be suitable for use with the present approach include parylene deposition, polytetrafluoroethylene (PTFE) coating, polyethylene glycol coating, polymer dip coating, or vacuum deposition (such as plasma-enhanced chemical vapor deposition (PECVD)).

With the preceding in mind, the present application utilizes a wire bonding approach to fabricate probes 54 onto a probe array 50. As may be appreciated, such wire bonding approaches are solid-phase welding processes by which an electrical interconnection is formed using thin wire and one or more of heat, pressure, and/or ultrasonic energy. In accordance with the present approach, wire bonded probe arrays 50 may be formed at room temperature, allowing probes 50 to be formed without subjecting the sensing platform to temperature excursions. In implementations where above-room temperature heating is employed, such heating is typically below 150° C.

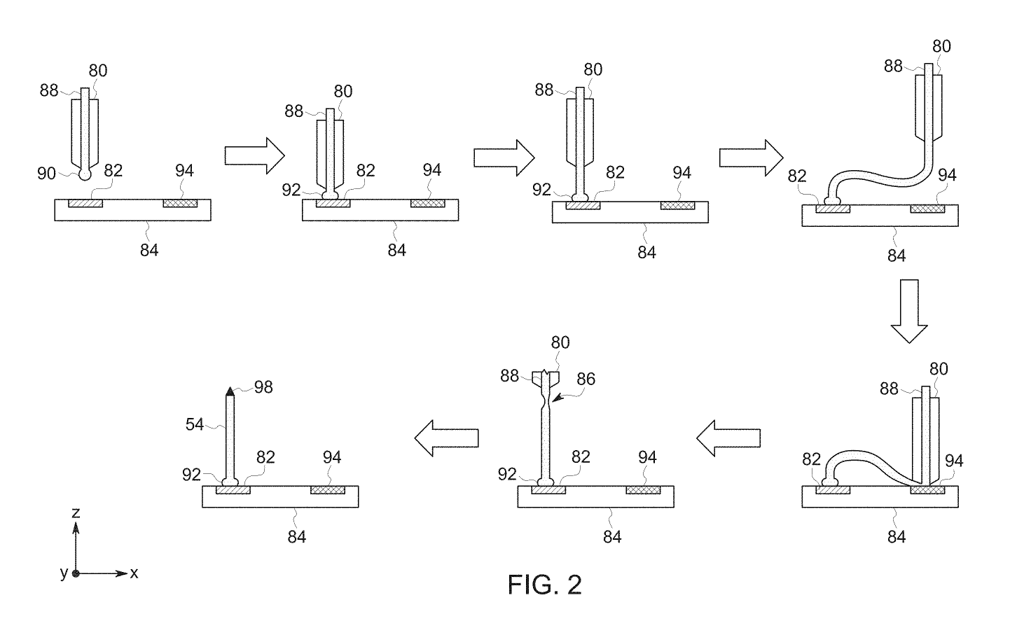

Turning to the visual process flow depicted in FIG. 2, in a wire bonding operation, a wire bonding tool 80 is positioned over an initial bond location 82. The initial bond location (e.g., a bonding pad 82) is formed on a substrate 84 and electrically connects to circuitry that will be used to electrically interact with the probe 54 to be formed on the pad 82. For example, the bond pad 82 may conductively connect to driving or readout circuitry by either topside circuit traces or through vias through the substrate 84 that conductively connect to circuitry on the backside of the substrate 84 (i.e., the surface opposite the bond pad 82).

The wire bonding tool 80 conventionally is referred to as a capillary and may take the form of a ceramic tool (shown in cross section) having a cylindrical cavity through which a wire 88 passes (i.e., is fed) during operation. In the depicted example, at the beginning of the operation (upper left corner of FIG. 2) a portion of wire 88 protruding from the tip of the wire bonding tool 80 has the shape of a sphere or ball 90, which may be due to an electrical spark or other heating event applied to the tip of the protruding wire 88 prior to a bonding operation.

As shown in the first two steps of FIG. 2, a bond is formed between the ball 90 and the bond pad 82 by lowering the wire bonding tool 80 to contact the bond location 82. Once in contact, thermal and/or ultrasonic energy is applied to the ball 90, forming a diffusion bond with the bond location 82. This initial bond may be referred to as a ball bond 92. Conventionally, the wire bonding tool 80 may then undergo a looping or “stitching” motion to create a loop of the wire 88 between the bond pad 82 and a second bonding location, here target region 94. This is shown in the third, fourth and fifth step of FIG. 2, where the wire bonding tool 80 lifts from the initial pad 82 trailing the bonded wire 88, moves laterally to be over the target region 94, and descends to contact the wire 88 to the target region 94.

In a conventional wire-bonding application, the target region 94 would comprise a material with which the material of the wire 88 would bond, such as to form a stitch- or wedge-bond. In the present approach, however, the target region 94 has a composition with which the wire material does not bond, such as silicon, glass, or a non-metallic passivation layer, such as SiO₂. Though depicted as a separate region in FIG. 2, the target region 94 may simply be the substrate 84 in embodiments where the wire material does form a bond with the substrate material.

As shown at the fifth step in the example, the wire bond tool 80 contacts the wire 88 over the non-bonding contact region 94. When so positioned, a tapered tip of the wire bond tool 80 may deform (e.g., crush or pinch) a portion of the wire 88 against the target region so as to form a tapered or thinned region 86 of the wire 88. In the present approach, as no bond is formed at target region 94, the wire-bond tool 80 may lift the wire 88, including thinned region 86, to a substantially vertical orientation (e.g., ninety-degrees relative to the surface of the substrate 84 plus or minus manufacturing tolerances) over the initial bond location 82, as shown in the sixth step of FIG. 2. As shown in the final step, the wire bond tool 80 may then be further lifted away from the substrate 84 to apply tension to the wire 88 and causing the wire 88 to snap or break at the thinned region 86 to form a probe 54 of a probe array. As shown with respect to FIG. 1, certain of the probes 54 may be made differing lengths so as to prevent an effectively uniform surface being formed by the tips of the probes 54. Such differing lengths may result from a longer “stitch” or loop of wire material being drawn from the initial pad location 82 to a target region 94.

In the depicted example, the tip 98 of the probe 54 has a wedge or conical shape, which may be characteristic of the act of breaking the wire 88 at the thinned region 86. The wedge or conical shape of the tip 98 may be conducive to insertion of the probe 54 into tissue. If such a sharpened or tapered shape is not generated by the wire cutting process, it may be achieved by etching or otherwise treating the probe tips 98 in a separate step of the manufacturing process.

As may be appreciated, the tip 98 exposed by the cutting and/or etching step that forms the probe 54 may also expose a conductive region (e.g., a gold wire core) of the probe, such as where the wire 88 is a dielectric coated wire with a conductive core. Correspondingly, the tip 98 of the probe 54 where the interior wire is exposed is conductive while the coated remainder of the probe 54 is not conductive or of limited conductivity.

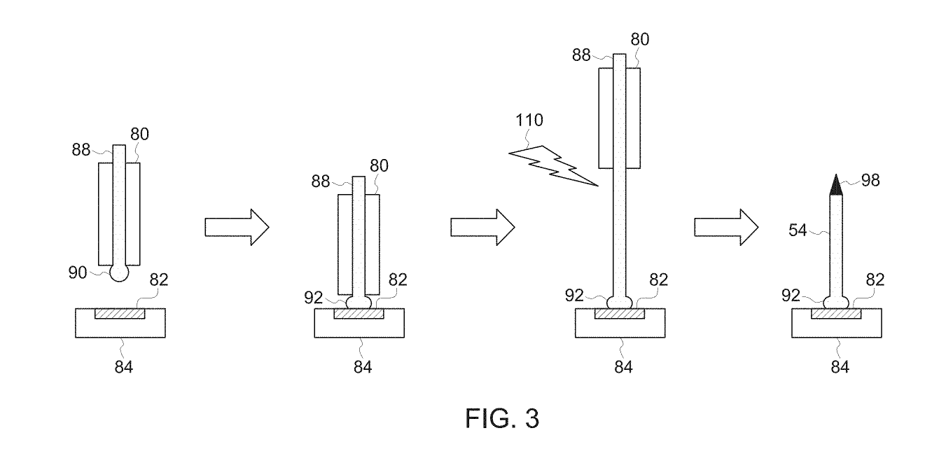

While FIG. 2 visually depicts one possible process flow for forming a probe 54 of a probe array 50 (e.g., a 10×10 array), FIG. 3 depicts an alternative process flow. In accordance with this process flow, the initial ball bond 92 is formed as discussed previously, as shown in the first two steps. However, instead of looping to a second target region, in the third step the wire bond tool 80 instead lifts upward to a specified height, as which point the wire 88 is cut using a suitable application of a thermal, electrical, or chemical event 110. The probe 54 is thereby formed, as shown in the final step. The event 110 may sever the wire 88 in such a way as to provide a sharpened (e.g., wedge-shaped or conical) tip 98 or such shaping may be applied subsequently, such as by an etching technique. Similarly, in embodiments in which the wire 88 is coated or otherwise has a conductive core and non-conductive sheathing, the event 110 or a subsequent etching may expose the conductive material at the tip 98. Further, as noted above, the height of different probes 54 formed on an array 50 using this approach may vary so as to present a non-uniform insertion surface on tissue, thereby minimizing or eliminating tenting effects.

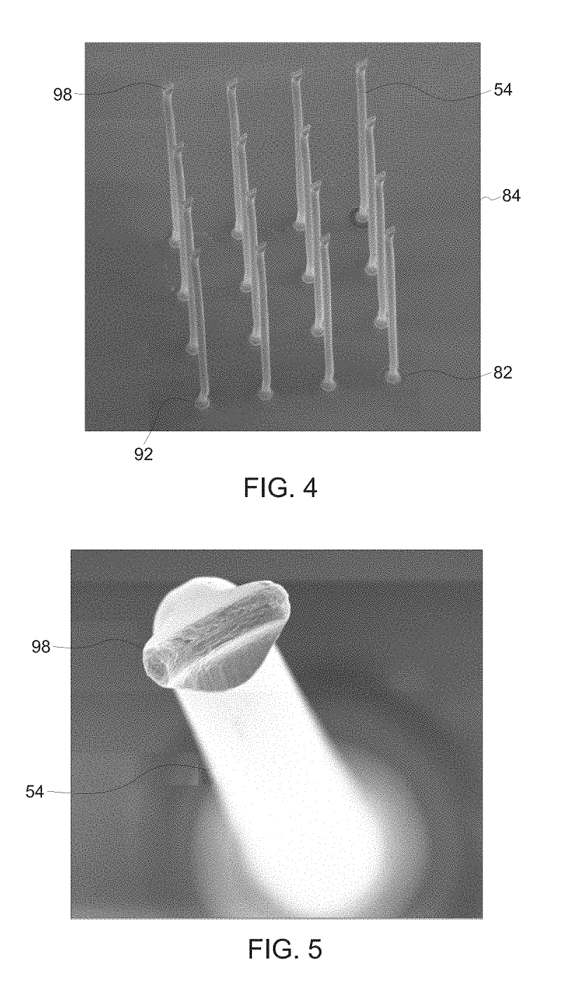

With the preceding in mind, FIGS. 4 and 5 are scanning electron micrographs of an array of probes 54 fabricated in accordance with the present approach. FIG. 4 depicts a 4×4 array of probes 54 formed on a probe array substrate 84. FIG. 5 shows a close-up view of a tip 98 of one of the probes 54, illustrating a tapered tip.

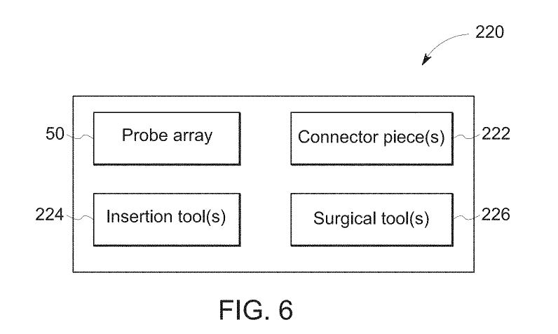

With the preceding probe array discussion in mind, FIG. 6 depicts an example of a kit 220 that may be provided for a respective medical or diagnostic procedure. In the depicted example, the kit 220 includes a probe array 50. The probe array 50 may be of a standardized configuration or may, in some implementations, be customized or tailored to an individual patient and/or procedure. The kit 220 may also include one or more connectors 222 suitable for connecting the probe array 50 to one or more respective medical devices, such as a monitor or other device suitable for reading signals from the probe array 50 and/or activating or powering the probes 54 of the probe array 50. In the depicted example, the kit 200 also includes one or more surgical tools 226 that may be provided to facilitate a surgical operation or procedure involving the probe array 50. Similarly, one or more insertion tools 224 may be provided as part of the kit 220 that may be used to facilitate the insertion or attachment of a probe array 50 relative to a target tissue site. As may be appreciated, one or more included pieces of the kit 220 may be provided as a single-use or disposable unit. For example, one or more of the probe array 50, connectors 222, insertion tools 224, and surgical tools 226 may be used once for a given procedure and then discarded.

Technical effects of the invention include fabricating probes of a probe array device using a wire bonding apparatus. In accordance with this approach, the wire bond apparatus bonds ones end of a wire to a region of a probe array substrate. The second end, however, is not bonded to the substrate and instead is either fabricated to be vertical with respect to the substrate or raised from a non-bonded site to be vertical with respect to the substrate. The process may be repeated to form multiple probes of the probe array.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of fabricating a probe of a probe array device, comprising:
lowering a wire bonding tool through which a wire passes toward a bonding pad of a probe array substrate;
forming a ball bond between an end of the wire and the bonding pad;
moving the wire bonding tool to a second region of the probe array substrate so that the wire extends from the ball bond to the second region;
deforming the wire at the second region to form a thinned region of the wire; and
moving the wire bonding tool so that the wire is at a substantially vertical orientation with respect to the bonding pad;
lifting the wire bonding tool so as to break the wire at the thinned region to form the probe of the probe array device.

2. The method of claim 1, wherein the wire comprises a conductive core and dielectric sheath.

3. The method of claim 1, wherein the wire comprises a dielectric coated gold or platinum wire.

4. The method of claim 1, wherein the wire has a diameter in the range of 10 μm to 75 μm.

5. The method of claim 1, wherein the probe has a length in the range of 50 μm to 1 cm.

6. The method of claim 1, wherein the wire bonding tool comprises a ceramic capillary having a cylindrical cavity through which the wire passes.

7. The method of claim 1, wherein a tip of the probe tip is tapered.

8. The method of claim 1, wherein a tip of the probe is conductive and a shaft of the probe is electrically isolated.

9. The method of claim 1, further comprising:
coating a tip of the probe with one or more of a functionalized polymer, oligomer, short chain material, ionic membrane, ionophore, enzyme, polycrystaline diamond, carbon coating, conductive polymer, carbon nanotubes, or metal alloy surface.

10. A method of fabricating a probe of a probe array device, comprising:
lowering a wire bonding tool through which a wire passes toward a bonding pad of a probe array substrate;
forming a ball bond between an end of the wire and the bonding pad;
moving the wire bonding tool away from the substrate in a direction perpendicular to a surface of the substrate on which the binding pad is located so that the wire extends outward perpendicular from the ball bond; and
forming a break in the wire to form a probe of a probe array, wherein the location of the break corresponds to a probe tip of the probe.

11. The method of claim 10, wherein the wire comprises a conductive core and dielectric sheath.

12. The method of claim 10, wherein the wire has a diameter in the range of 10 μm to 75 μm.

13. The method of claim 10, wherein the probe has a length in the range of 50 μm to 1 cm.

14. The method of claim 10, wherein the probe tip is tapered.

15. The method of claim 10, wherein the probe tip is conductive and a shaft of the probe is electrically isolated.

16. The method of claim 10, further comprising:
coating the probe tip with one or more of a functionalized polymer, oligomer, short chain material, ionic membrane, ionophore, enzyme, polycrystaline diamond, carbon coating, conductive polymer, carbon nanotubes, or metal alloy surface.

17. A neural probe array, comprising:
a probe array substrate;
a plurality of bonding pads formed on the probe array substrate; and
on each bonding pad, a respective wire bonded to each respective bonding pad to form a probe, wherein each probe is oriented substantially perpendicular to the probe array substrate.

18. The neural probe array of claim 17, wherein each wire comprise a conductive core and dielectric sheath.

19. The neural probe array of claim 17, wherein each wires have a diameter in the range of 10 μm to 75 μm.

20. The neural probe array of claim 17, wherein each wire is bonded to the respective bonding pads with ball bonds.
